(19) United States
(12) Patent Application Publication
(10) Pub. No.: US 2014/0073691 A1
(43) Pub. Date: Mar. 13, 2014

(54) Methods and composition for Treating Thyroid Hormone Related Disorders

(71) Applicant: Thomas E. Hughes, Boston, MA (US)

(72) Inventor: Thomas E. Hughes, Boston, MA (US)

(73) Assignee: Zafgen, Inc., Cambridge, MA (US)

(21) Appl. No.: 13/884,110

(22) PCT Filed: Nov. 10, 2011
(86) PCT No.: PCT/US2011/060127

§ 371 Date: Nov. 19, 2013

Related U.S. Application Data
(60) Provisional application No. 61/412,008, filed on Nov. 10, 2010.

(51) Int. Cl. A61K 31/336; A61K 45/06

(52) U.S. Cl. CPC A61K 31/336; A61K 45/06
USPC 514/475

(57) ABSTRACT

The invention generally relates to methods of improving thyroid hormone sensitivity or treating a thyroid hormone resistance related disorder in a patient in need thereof, comprising administering a pharmaceutically effective amount of a MetAP-2 inhibitor. Such methods may include administering a MetAP-2 inhibitor at a dose that does not substantially modulate angiogenesis.

### CROSS REFERENCE TO RELATED APPLICATIONS

This application claims priority to U.S. Provisional Patent Application 61/412,008, filed Nov. 10, 2010, and hereby incorporated by reference in its entirety.

### BACKGROUND

Chronic metabolic debilitation syndromes such as fibromyalgia and chronic fatigue syndrome are typically associated with widespread musculoskeletal pain, immune dysfunction, neurotransmitter dysfunction, joint stiffness, fatigue, gastrointestinal distress and urinary distress, cognitive dysfunction and/or other neurological symptoms. It has been further reported that these syndromes, such as fibromyalgia are associated with difficulties in thyroid production or utilization, e.g., hypothyroidism. (Garrison and Breeding, Med. Hypotheses (2003) 61(2), 182-189).

Hypothyroidism may be categorized into type I (hormone deficient) and type II (hormone resistant). Fibromyalgia is associated with type II hypothyroidism and dysfunction of the hypothalamic-pituitary-thyroid and hypothamlamic-pituitary-adrenal axes (Neeck and Riedel, J Rheumatol (1992) 19(7: 112-2; Kadetoff and Kosek, J. Rehalib. Med. (2010)42(8):765-72), and it has been hypothesized that patients suffering from other chronic metabolic debilitation syndromes such as chronic fatigue syndrome, Gulf war syndrome, premenstrual syndrome, post traumatic stress disorder, breast implant silicone sensitivity syndrome, bipolar affective disorder, systemic candidiasis, myofasical pain syndrome, and/or idiopathic environmental intolerance, which typically have symptom overlap with fibromyalgia, also have disorders of thyroid utilization.

Fibromyalgia is a frequent cause of chronic, widespread pain, and is characterized by a generalized heightened perception of sensory stimuli. Patients with fibromyalgia display abnormalities in pain perception and may have other associated symptoms including fatigue, nonrestorative sleep, and memory difficulties. Chronic fatigue syndrome is a debilitating disorder characterized by profound tiredness or fatigue. Patients with chronic fatigue syndrome may become exhausted with only light physical exertion, and must often function at a level of activity substantially lower than their capacity before the onset of illness. In addition to the key defining characteristic of fatigue, chronic fatigue patients generally report various nonspecific symptoms, including weakness, muscle aches and pains, excessive sleep, malaise, fever, sore throat, tender lymph nodes, impaired memory and/or mental concentration, insomnia, and depression.

Obese, and to some extent, overweight patients may have an even higher risk of diseases such as fibromyalgia. (Mork, Arthr Care Res. (2010); 62:611-617) For example, obese women may have 70% or higher risk for fibromyalgia, as compared to normal weight women with similar activity levels.

Although a broad array of medications are used for chronic metabolic debilitation syndromes, no single pharmacological agent or combination of agents is effective in the treatment of these disorders. Thus, due to the lack of effective treatment regimens, there is a need to develop effective treatments, especially obese and/or diabetic patients.

### SUMMARY

At least in part, this disclosure is directed to a method of improving thyroid hormone sensitivity or treating a thyroid hormone sensitivity related disorder in a patient in need thereof is comprising administering a pharmaceutically effective amount of a MetAP-2 inhibitor.

Such thyroid hormone sensitivity related disorders may be selected from the group consisting of: fibromyalgia, chronic fatigue syndrome, Gulf war syndrome, premenstrual syndrome, post traumatic stress disorder, breast implant silicone sensitivity syndrome, bipolar affective disorder, systemic candidiasis, myofasical pain syndrome, and idiopathic environmental intolerance.

MetAP2 inhibitors may be a substantially irreversible inhibitor, e.g. fumagillin, fumagillol or fumagillin ketone, or derivatives thereof, siRNA, shRNA, an antibody, or a antisense compound, or may be a substantially reversible inhibitor. For example, an MetAP2 inhibitor may be selected from O-(4-dimethylaminoethoxycinnamoyl)fumagillol and pharmaceutically acceptable salts thereof.

### BRIEF DESCRIPTION OF FIGURES

FIG. 1 depicts the results of administration of a MetAP2 inhibitor to obese patients and indicates the reduction in plasma thyroid stimulating hormone, without reductions in circulating thyroid hormone (free T3 and T4) concentrations.

### DETAILED DESCRIPTION

### Overview

The disclosure relates at least in part to methods for treating a patient improving thyroid hormone sensitivity or treating a thyroid hormone sensitivity related disorder. For example, provided herein are methods of treating a chronic metabolic debilitation syndrome such as fibromyalgia or chronic fatigue syndrome in a patient in need thereof, which include administering an effective amount of a MetAP2 inhibitor. Also provided herein is a method of reducing thyroid stimulating hormone in a patient comprising administering MetAP-2 inhibitors, such as disclosed herein. In some embodiments, such administration may increase circulating thyroid hormones T3 and/or T4 owing to improved sensitivity of thyroid gland sensitivity to thyroid hormone stimulating hormones. In other embodiments, such administration may not significantly or substantially affect circulating thyroid hormones T3 and/or T4.

MetAP2 encodes a protein that functions at least in part by enzymatically removing the amino terminal methionine residue from certain newly translated proteins. Increased expression of the MetAP2 gene has been historically associated with various forms of cancer. Molecules inhibiting the enzymatic activity of MetAP2 have been identified and have been explored for their utility in the treatment of various tumor types and infectious diseases such as microsporidiosis, leishmaniasis, and malaria. However, diseases that are treatable by improving thyroid hormone sensitivity, e.g., by treatments that improve thyroid function or status while for example reducing thyroid stimulating hormone, such as fibromyalgia (and wherein such patients may be known to have a hypothyroid problem and/or elevated thyroid stimulating hormone levels), are typically not a form of cancer, and it has been found that MetAP2 inhibitors can effectively reduce thyroid stimulating hormone levels in plasma of patients without substantially reducing plasma thyroid hormone concentrations. Disclosed herein are methods relating to administering a MetAP-2 inhibitor to treat thyroid hormone sensitivity disorders, e.g., by administering an effective amount of a MetAP-2 inhibitor, e.g. a therapeutically effective amount but that does not substantially modulate or suppress angiogenesis. Also contemplated herein are methods for treating thyroid hormone sensitivity disorders caused by viral, bacterial, microbial and/or fungal infections by administering a MetAP-2 inhibitor. In an embodiment, provided herein is a method for improving thyroid function in patients suffering from hypothyroidism and/or a method for reducing thyroid replacement requirements in patients currently in need of a thyroid hormone replacement therapy, comprising administering a MetAP-2 inhibitor.

### MetAP2 Inhibitors

MetAP2 inhibitors refer to a class of molecules that inhibit or modulate the activity of MetAP2, e.g., the ability of MetAP2 to cleave the N-terminal methionine residue of newly synthesized proteins to produce the active form of the protein, or the ability of MetAP2 to regulate protein synthesis by protecting the subunit of eukaryotic initiation factor-2 (eIF2) from phosphorylation.

Exemplary MetAP2 inhibitors may include irreversible inhibitors that covalently bind to MetAP2. For example, such irreversible inhibitors include fumagillin, fumagillol, and fumagillin ketone.

Derivatives and analogs of fumagillin, and pharmaceutically acceptable salts thereof are contemplated herein as irreversible MetAP2 inhibitors, such as O-(4-dimethylaminoethoxycinnamoyl)fumagillol (also referred to herein as Compound A), O-(3,4,5-trimethoxycinnamoyl)fumagillol, O-(4-chlorocinnamoyl)fumagillol; O-(4-aminocinnamoyl)fumagillol; O-(4-dimethylaminoethoxycinnamoyl)fumagillol; O-(4-methoxycinnamoyl)fumagillol; O-(4-dimethylaminocinnamoyl)fumagillol; O-(4-hydroxycinnamoyl)fumagillol; O-(3,4-dimethoxycinnamoyl)fumagillol; O-(3,4-methylenedioxycinnamoyl)fumagillol; O-(3,4,5-trimethoxycinnamoyl)fumagillol; O-(4-nitrocinnamoyl)fumagillol; O-(3,4-dimethoxy-6-aminocinnamoyl)fumagillol; O-(4-acetoxy-3,5-dimethoxycinnamoyl)fumagillol; O-(4-ethylaminocinnamoyl)fumagillol; O-(4-ethylaminoethoxycinnamoyl)fumagillol; O-(3-dimethylaminomethyl-4-methoxycinnamoyl)fumagillol; O-(4-trifluoromethylcinnamoyl)fumagillol; O-(3,4-dimethoxy-6-nitrocinnamoyl)fumagillol; O-(4-acetoxycinnamoyl)fumagillol; O-(4-cyanocinnamoyl)fumagillol; 4-(4-methoxycinnamoyl)oxy-2-(1,2-epoxy-1,5-dimethyl-4-hexenyl)-3-methoxy-1-chloromethyl-1-cyclohexanol; O-(3,4,5-trimethoxycinnamoyl)fumagillol; O-(4-dimethylaminocinnamoyl)fumagillol; O-(3 ,4,5-trimethoxycinnamoyl)oxy-2-(1,2-epoxy-1,5-dimethyl-4-hexenyl)-3-m-ethoxy-1-chloromethyl-1-cyclohexanol; O-(4-dimethylaminocinnamoyl)oxy-2-(1,2-epoxy-1,5-dimethyl-4-hexenyl)-3-me-thoxy-1-chloromethyl- 1-cyclohexanol; O-(3,5-dimethoxy-4-hydroxycinnamoyl)fumagillol or O-(chloracetyl-carbamoyl) fumagillol(TNP-470), and/or pharmaceutically acceptable salts thereof (e.g. O-(4-dimethylaminoethoxycinnamoyl)fumagillol oxalate).

Fumagillin, and some derivatives thereof, have a carboxylic acid moiety and can be administered in the form of the free acid. Alternatively, contemplated herein are pharmaceutically acceptable salts of fumagillin, fumagillol, and derivatives thereof.

Pharmaceutically acceptable salts illustratively include those that can be made using the following bases: ammonia, L-arginine, benethamine, benzathene, betaine, bismuth, calcium hydroxide, choline, deanol, diethanolamine, diethylamine, 2-(diethylamino)ethanol, ethylenediamine, N-methylglucarnine, hydrabamine, 1 H-imidazole, lysine, magnesium hydroxide, 4-(2-hydroxyethyl)morpholine, piperazine, potassium hydroxide, 1-(2-hydroxyethyl)pyrrolidine, sodium hydroxide, triethanolamine, zinc hydroxide, diclyclohexlamine, or any other electron pair donor (as described in Handbook of Pharmaceutical Salts, Stan & Wermuth, VHCA and Wiley, Uchsenfurt-Hohestadt Germany, 2002). Contemplated pharmaceutically acceptable salts may include hydrochloric acid, bromic acid, sulfuric acid, phosphoric acid, nitric acid, formic acid, acetic acid, trifluoroacetic acid, oxalic acid, fumaric acid, tartaric acid, maleic acid, methanesulfonic acid, benzenesulfonic acid or para-toluenesulfonic acid.

Esters of the present invention may be prepared by reacting fumagillin or fumagillol with the appropriate acid under standard esterification conditions described in the literature (Houben-Weyl 4th Ed. 1952, Methods of Organic Synthesis). Suitable fumagillin esters include ethyl methanoate, ethyl ethanoate, ethyl propanoate, propyl methanoate, propyl ethanoate, and methyl butanoate.

In another embodiment, contemplated irreversible inhibitors of MetAP2 may include a siRNA, shRNA, an antibody or an antisense compound of MetAP2.

Further examples of reversible and irreversible MetAP2 inhibitors are provided in the following references, each of which is hereby incorporated by reference: Olson et al. (U.S. Pat. No. 7,084,108 and WO 2002/042295), Olson et al. (U.S. Pat. No. 6,548,477; U.S. Pat. No. 7,037,890; U.S. Pat. No. 7,084,108; U.S. Pat. No. 7,268,111; and WO 2002/042295), Olson et al. (WO 2005/066197), Hong et al. (U.S. Pat. No. 6,040,337)., Hong et al. (U.S. Pat. No. 6,063,812 and WO 1999/059986), Lee et al. (WO 2006/080591), Kishimoto et al. (U.S. Pat. No. 5,166,172; U.S. Pat. No. 5,698,586; U.S. Pat. Nos. 5,164,410; and 5,180,738), Kishimoto et al. (U.S. Pat. No. 5,180,735), Kishimoto et al. (U.S. Pat. No. 5,288,722), Kishimoto et al. (U.S. Pat. No. 5,204,345), Kishimoto et al. (U.S. Pat. No. 5,422,363), Liu et al. (U.S. Pat. No. 6,207,704; U.S. Pat. No. 6,566,541; and WO 1998/056372), Craig et al. (WO 1999/057097), Craig et al. (U.S. Pat. No. 6,242,494), BaMaung et al. (U.S. Pat. No. 7,030,262), Comess et al. (WO 2004/033419), Comess et al. (U.S. 2004/0157836), Comess et al. (U.S. 2004/0167128), Henkin et al. (WO 2002/083065), Craig et al. (U.S. Pat. No. 6,887,863), Craig et al. (U.S. 2002/0002152), Sheppard et al. (2004, Bioorganic & Medicinal Chemistry Letters 14:865-868), Wang et al. (2003, Cancer Research 63:7861-7869), Wang et al. (2007, Bioorganic & Medicinal Chemistry Letters 17:2817-2822), Kawai et al. (2006, Bioorganic & Medicinal Chemistry Letters 16:3574-3577), Henkin et al. (WO 2002/026782), Nan et al. (U.S. 2005/0113420), Luo et al. (2003, J. Med. Chem., 46:2632-2640), Vedantham et al. (2008, J. Comb. Chem., 10:195-203), Wang et al. (2008, J. Med. Chem., 51 (19):6110-20), Ma et al. (2007, BMC Structural Biology, 7:84) and Huang et al. (2007, J. Med. Chem., 50:5735-5742), Evdokimov et al. (2007, PROTEINS: Structure, Function, and Bioinformatics, 66:538-546), Garrabrant et al. (2004, Angiogenesis 7:91-96), Kim et al. (2004, Cancer Research, 64:2984-2987), Towbin et al. (2003, The Journal of Biological Chemistry, 278(52):52964-52971), Marino Jr. (U.S. Pat. No. 7,304,082), Kallender et al. (U.S. patent application No. 2004/0192914), and Kallender et al. (U.S. patent application Nos. 2003/0220371 and 2005/0004116). Other MetAP2 inhibitors contemplated herein are disclosed in U.S. Ser. Nos. 61/310,776; 61/293,318; 61/366,650 and PCT/US10/52050 (all of the above are hereby incorporated by reference in their entirety).

For example, contemplated MetAP2 inhibitors may include:

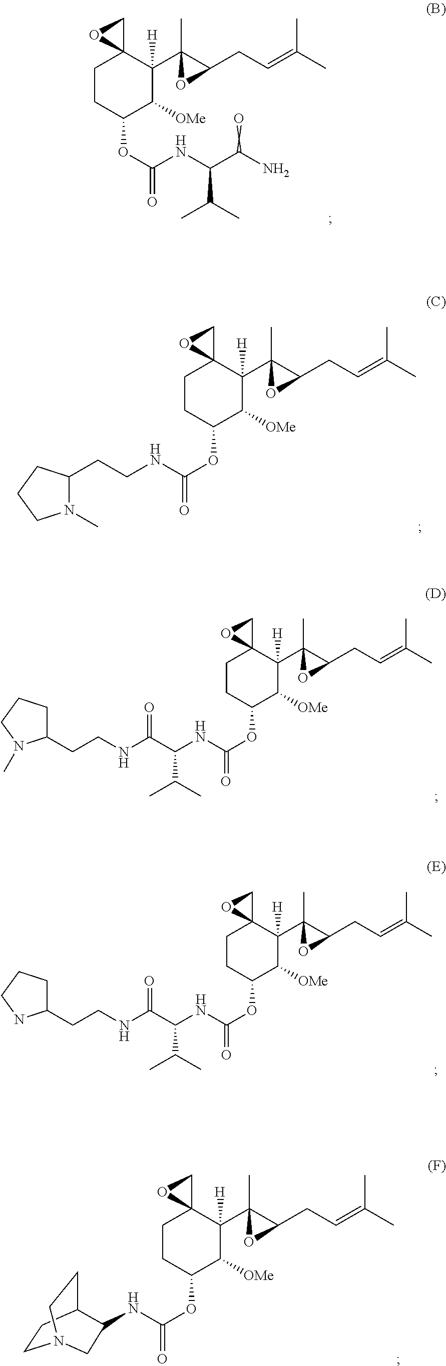

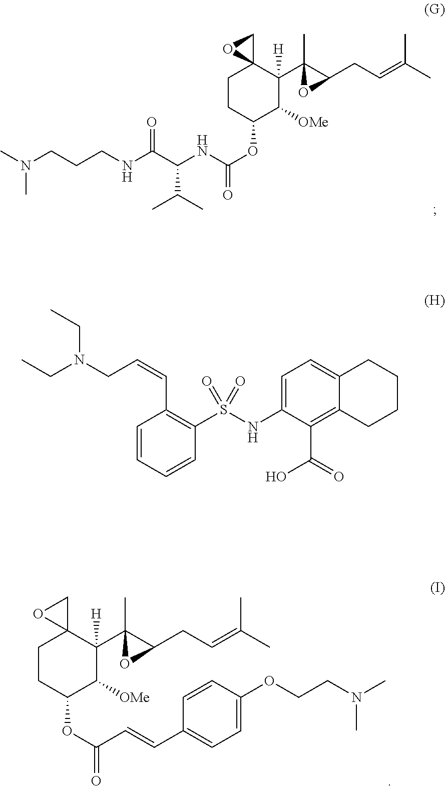

### Methods

A method of improving thyroid hormone sensitivity or treating a thyroid hormone sensitivity related disorder (such as a chronic metabolic debilitation syndrome) in a patient in need thereof is provided herein that comprising administering a pharmaceutically effective amount of a MetAP-2 inhibitor such as those provided herein. Such thyroid hormone sensitivity related disorders may be selected from the group consisting of: fibromyalgia, chronic fatigue syndrome, Gulf war syndrome, premenstrual syndrome, post traumatic stress disorder, breast implant silicone sensitivity syndrome, bipolar affective disorder, systemic candidiasis, myofasical pain syndrome, hypothyroidism, and/or idiopathic environmental intolerance.

In some embodiments, a contemplated method includes also administering a thyroid hormone replacement therapy to the patient, e.g., administering a thyroid hormone. In some embodiments, a patient may be currently taking a thyroid hormone replacement therapy, and upon administration of said Met-AP2 inhibitor, the thyroid hormone replacement therapy is reduced in dose, e.g., there is a reduction in the amount of needed hormone replacement upon administration of a Met-AP2 inhibitor.

In an embodiment, a method of treating fibromyalgia in a patient in need thereof is provided comprising administering a pharmaceutically effective amount of a MetAP-2 inhibitor. Contemplated pharmaceutically effective amounts may not, in some embodiments, substantially modulate or suppress angiogenesis.

Patients contemplated for treatment of disclosed disorders or diseases include patients of normal weight, or an obese patient and/or a patient suffering from diabetes. In some embodiments, patients contemplated for treatment include those who are suffering from a thyroid hormone sensitivity related disorder and/or may have an elevated circulating thyroid hormone level, for example, a circulating thyroid hormone level of greater than about 2.5 mIU/L.

Also provided herein is a method of improving thyroid hormone resistance, thereby reducing thyroid stimulating hormone in the plasma of a patient in need thereof, comprising administering a pharmaceutically effective amount of a MetAP-2 inhibitor. In some embodiments, upon administration, plasma levels of circulating thyroid hormones T3 and T4 are not significantly abnormal in a patient.

In some embodiments, co-administration of a MetAP-2 inhibitor and another active agent (e.g. an analgesic, and/or a thyroid hormone) occur at the same time. In other embodiments, administration of a MetAP-2 inhibitor occurs immediately prior to or immediately administration of another active agent. In yet another embodiment, a period of time may elapse between administration of a MetAP-2 inhibitor and another agent. For example, methods disclosed herein may include co-administration of a MetAP-2 inhibitor and a tricyclic or tetracyclic anti-depressant agent (such as amitripytline, amitriptylinoxide, butriptyline, clomipramine, demexiptiline, desipramine, dibenzepin, dimetacrine, dosulepin, doxepin, imipramine, imipraminoxide, lofepramine, melitracen, metapramine, nitroxazepine, nortripyline, noxiptiline, pipofezine, propizepine, protriptyline and quinupramine. Using such co-administration, the MetAP-2 inhibitor may provide both improvement of the thyroid hormone resistant related dysfunction, and also inhibit pharmacological weight gain caused by the anti-depressant or other agent.

### Administration and Formulation

Contemplated herein are formulations suitable for parenteral or non-parenteral administration of MetAP2 inhibitors. In certain embodiments, a subject may have a lower systemic exposure (e.g. at least about 2, 3, 5, 10, 20, or at least about 30% less systemic exposure) to the non-parenterally (e.g. orally) administered of a MetAP2 inhibitor as compared to a subject parenterally (e.g. subcutaneously) administered the same dose of the MetAP2 inhibitor.

Contemplated non-parenteral administration includes oral, buccal, transdermal (e.g. by a dermal patch), topical, inhalation, sublingual, ocular, pulmonary, nasal, or rectal administration.

Contemplated parenteral administration includes intravenous and subcutaneous administration, as well as administration at a site of a minimally-invasive procedure or a surgery.

In another embodiment, provided herein are effective dosages, e.g. a daily dosage of a MetAP2 inhibitor, that may not substantially modulate or suppress angiogenesis. For example, provided here are methods that include administering doses of MetAP2 inhibitors that are effective for e.g. reducing thyroid stimulating hormone and associated condition, but are significantly smaller doses than that necessary to modulate and/or suppress angiogenesis (which may typically require about 12.5 mg/kg to about 50 mg/kg or more). For example, contemplated dosage of a MetAP2 inhibitor in the methods described herein may include administering about 25 mg/day, about 10 mg/day, about 5 mg/day, about 3 mg/day, about 2 mg/day, about 1 mg/day, about 0.75 mg/day, about 0.5 mg/day, about 0.1 mg/day, about 0.05 mg/day, or about 0.01 mg/day.

For example, an effective amount of the drug for reducing thyroid stimulating hormone and improving or ameliorating the associated condition in a patient in need thereof may be about 0.0001 mg/kg to about 25 mg/kg of body weight per day. For example, a contemplated dosage may from about 0.001 to 10 mg/kg of body weight per day, about 0.001 mg/kg to 1 mg/kg of body weight per day, about 0.001 mg/kg to 0.1 mg/kg of body weight per day or about 0.005 to about 0.04 mg/kg or about 0.005 to about 0.049 mg/kg of body weight a day. In an embodiment a MetAP2 inhibitor such as disclosed herein (e.g. O-(4-dimethlyaminoethoxycinnamoyl)fumagillol), may be administered about 0.005 to about 1 mg/kg, or to about 5 mg/kg, or about 0.005 to about 0.1 mg/kg of a subject.

For example, provided herein is a method for treating or reducing the risk of a thyroid hormone resistance syndrome-associated condition in a subject in need thereof, comprising administering, parenterally (e.g. intravenously) or non-parenterally, about 0.005 to about 1 mg/kg, or about 0.005 to about 1.0 mg/kg or to 0.005 to about 0.05 mg/kg of a MetAP2 inhibitor, selected from O-(4-dimethylaminoethoxycinnamoyl)fumagillol and pharmaceutically acceptable salts thereof (for example, an oxalate salt), to said subject.

Contemplated methods may include administration of a composition comprising a MetAP2 inhibitor, for example, hourly, twice hourly, every three to four hours, daily, twice daily, 1, 2, 3 or 4 times a week, every three to four days, every week, or once every two weeks depending on half-life and clearance rate of the particular composition or inhibitor and/or the duration of its action.

Treatment can be continued for as long or as short a period as desired. The compositions may be administered on a regimen of, for example, one to four or more times per day. A suitable treatment period may be, for example, at least about one week, at least about two weeks, at least about one month, at least about six months, at least about 1 year, or indefinitely. A treatment regimen may include a corrective phase, during which a MetAP2 inhibitor dose sufficient to provide e.g., reduction of symptoms is administered, followed by a maintenance phase, during which a lower MetAP2 inhibitor dose sufficient to reduce or prevent increase in occurrence of symptoms of the treated disease is administered.

For pulmonary (e.g., intrabronchial) administration, MetAP2 inhibitors may be formulated with conventional excipients to prepare an inhalable composition in the form of a fine powder or atomizable liquid. For ocular administration, MetAP2 inhibitors may be formulated with conventional excipients, for example, in the form of eye drops or an ocular implant. Among excipients useful in eye drops are viscosifying or gelling agents, to minimize loss by lacrimation through improved retention in the eye.

Liquid dosage forms for oral or other administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition may include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, cutaneous routes of administration are achieved with aqueous drops, a mist, an emulsion, or a cream.

Transdermal patches may have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

When administered in lower doses, injectable preparations for intravenous or subcutaneous administration are also contemplated herein, for example, sterile injectable aqueous or oleaginous suspensions may be formulated according to the known art using suitable dispersing or wetting agents and suspending agents.

Compositions for rectal administration may be suppositories which can be prepared by mixing a MetAP2 inhibitor with suitable non-irritating excipients or carriers such as cocoa butter, polyethylene glycol or a suppository wax which are solid at ambient temperature but liquid at body temperature and therefore melt in the rectum and release the active agent(s). Alternatively, contemplated formulations can be administered by release from a lumen of an endoscope after the endoscope has been inserted into a rectum of a subject.

Oral dosage forms, such as capsules, tablets, pills, powders, and granules, may be prepared using any suitable process known to the art. For example, a MetAP2 inhibitor may be mixed with enteric materials and compressed into tablets.

Alternatively, formulations of the invention are incorporated into chewable tablets, crushable tablets, tablets that dissolve rapidly within the mouth, or mouth wash.

### EXAMPLES

The following examples are not intended in any way to limit the scope of this invention but is provided to illustrate aspects of the disclosed methods. Many other embodiments of this invention will be apparent to one skilled in the art.

### Example 1

### Administration of MetAP-2 Inhibitor Reduces Thyroid Stimulating Hormone (TSH)

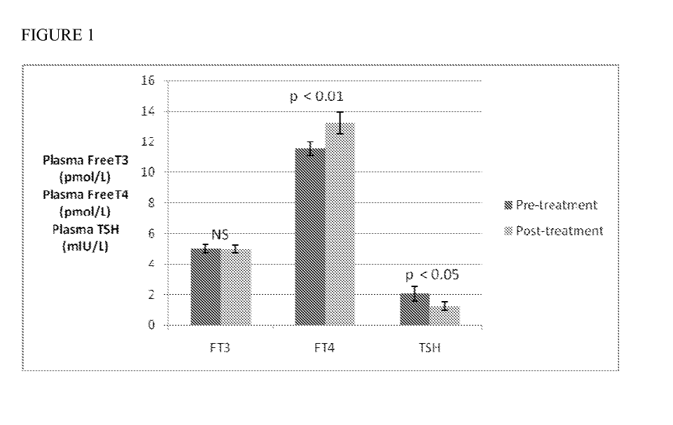

8 obese subjects who were not taking any thyroid medication were treated with O-(4-dimethylaminoethoxycinnamoyl)fumagillol at a dose of 0.9 mg per square meter of body surface area (approximately 1.8 mg per dose) for 26 days. A significant reduction in plasma thyroid stimulating hormone (TSH) without a reduction in circulating thyroid hormones T3 and T4 was observed. (FIG. 1). This is indicative of improved thyroid hormone sensitivity and a reduction in thyroid hormone resistance.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of improving thyroid hormone sensitivity or treating a thyroid hormone sensitivity related disorder in a patient in need thereof, comprising administering a pharmaceutically effective amount of a MetAP-2 inhibitor.

2. The method of claim 1, wherein the thyroid hormone sensitivity related disorder is selected from the group consisting of: fibromyalgia, chronic fatigue syndrome, Gulf war syndrome, premenstrual syndrome, post-traumatic stress disorder, breast implant silicone sensitivity syndrome, bipolar affective disorder, systemic candidiasis, myofasical pain syndrome, hypothyroidism, and idiopathic environmental intolerance.

3. The method of claim 1, wherein a thyroid hormone replacement therapy is also administered to the patient.

4. The method of claim 1, wherein the patient is currently administered a thyroid hormone replacement therapy, and upon administration of said Met-AP2 inhibitor, the thyroid hormone replacement therapy is reduced in dose.

5. The method of claim 3, wherein the thyroid hormone therapy is a thyroid hormone.

6. A method of treating chronic metabolic debilitation syndrome in a patient in need thereof, comprising administering a pharmaceutically effective amount of a MetAP-2 inhibitor.

7. A method of treating fibromyalgia in a patient in need thereof, comprising administering a pharmaceutically effective amount of a MetAP-2 inhibitor.

8. The method of claim 1, wherein said pharmaceutically effective amount does not substantially modulate or suppress angiogenesis.

9. The method of claim 1, wherein the patient is obese.

10. The method of claim 1, wherein the patient is suffering from diabetes.

11. A method of reducing thyroid stimulating hormone in the plasma of a patient in need thereof, comprising administering a pharmaceutically effective amount of a MetAP-2 inhibitor, wherein upon administration, plasma levels of circulating thyroid hormones T3 and T4 are not significantly reduced.

12. The method of claim 1, wherein said MetAP2 inhibitor is selected from the group consisting of fumagillin, fumagillol, or a fumagillin ketone derivative, siRNA, shRNA, an antibody, or an antisense compound.

13. The method of claim 1, wherein said MetAP2 inhibitor is selected from O-(4-dimethylaminoethoxycinnamoyl)fumagillol and pharmaceutically acceptable salts thereof.

14. The method of claim 1, wherein said MetAP2 inhibitor is a substantially reversible inhibitor.

15. The method of claim 1, wherein said MetAP2 inhibitor is administered parenterally or non-parenterally.

16. The method of claim 1, further comprising administering an analgesic and/or an anti-depressant.
